(19) United States
(12) Patent Application Publication
(10) Pub. No.: US 2021/0113740 A1
(43) Pub. Date: Apr. 22, 2021

(54) DECELLULARIZED CORNEAL MATRIX BASED HYDROGEL, BIOINK FORMULATION AND METHODS THEREOF

(71) Applicant: INDIAN INSTITUTE OF TECHNOLOGY HYDERABAD, Telangana (IN)

(72) Inventors: Shibu CHAMEETTACHAL, Telangana (IN); Falguni PATI, Telangana (IN)

(73) Assignees: INDIAN INSTITUTE OF TECHNOLOGY HYDERABAD, Telangana (IN); HYDERABAD EYE RESEARCH FOUNDATION, Telegana (IN)

(21) Appl. No.: 16/981,957

(22) PCT Filed: Mar. 16, 2019
(86) PCT No.: PCT/IB2019/052143

§ 371 Date: Sep. 17, 2020

(30) Foreign Application Priority Data
Mar. 17, 2018 (IN) 201841009835

(51) Int. Cl. A61L 27/52; A61L 27/36; A61L 27/38

(52) U.S. Cl. CPC A61L 27/52; A61L 27/3687; A61L 27/3813; A61L 27/3834; A61L 27/3808; A61L 2430/16; A61L 27/3839; A61L 27/3641; A61L 27/3886; A61L 2430/40; A61L 27/3633

(57) ABSTRACT

Methods for preparation of decellularized corneal matrix-based hydrogel and bioink. The process involves decellularization of corneal samples, digestion of the corneal samples, adjustment of the pH and finally preparation of the hydrogel. The hydrogels produced by the method are extremely useful for treatment of various pathological conditions pertaining to cornea.

### FIELD OF INVENTION

The present invention relates to method for decellularization of corneal extracellular matrix and preparation of hydrogels for use in a number of diseases and medical conditions pertaining to cornea.

### BACKGROUND

Cornea is the transparent, dome-shaped covering at the front of eye which focuses light into retina and is the main refractive element of the eye. Corneal diseases are one of the major causes of blindness in the world. The diseases are characterized by loss of corneal transparency, which subsequently deteriorates the vision.

Penetrating keratoplasty is the most widely accepted treatment for corneal blindness, which involves transplantation of a healthy donor cornea. Another important treatment of corneal diseases involves corneal collagen cross-linking with the use of riboflavin and ultraviolet in which cross-links are induced in the corneal stroma which produces a stiffening effect and increasing corneal strength and stability. Another successful surgical method is Bowman membrane transplantation, which is a technique used for strengthening the cornea in case of advanced keratoconus by means of mid-stromal transplantation of an isolated Bowman layer graft.

However, the current approaches used for dealing with medical conditions pertaining to cornea suffers from various limitations such as risk factor involved, cost and need for expertise. The aforementioned procedures are extremely risky as it can damage the Descemet's membrane and endothelial layer, which are impossible to repair. Further, techniques such as cross-linking with riboflavin or UV requires extremely skilled personnel, and there are chances of damage to the endothelial layer of the eye due to the presence of the highly reactive cross-linking agents. Finally, in techniques like penetrating keratoplasty, the supply of healthy donor corneas is not sufficient to meet the demands.

The aforementioned scenario coupled with factors such as low investment in healthcare (1.5% of GDP in India as compared to 10-18% in high income countries) and low ratio of medical personnel compared to number of patients leads to no treatment for a huge proportion of population in developing and less developed countries. Therefore, it becomes extremely necessary to provide an alternative solution which does not require a highly skilled surgeon for treatment of medical conditions pertaining to cornea.

Therefore, the present invention contemplates to provide compositions and methods for preparation of injectable hydrogels for use in a number of diseases and medical conditions pertaining to cornea. The present invention overcomes the problems of the prior art to solve a long-standing problem of providing compositions for inexpensive treatment of corneal disorders. The invention would facilitate the access to improved affordable hydrogels to the world's visually impaired specifically who live in low-income nations.

### SUMMARY OF THE INVENTION

### Technical Problem

The technical problem to be solved in this invention is providing an inexpensive and highly efficacious decellularized corneal matrix-based hydrogel for uses in a number of diseases and medical conditions pertaining to cornea.

### Solution to the Problem

The problem has been solved by a multi-dimensional approach involving devising a method for preparation of corneal matrix hydrogel. The process involves decellularization of corneal samples, digestion of the corneal samples, adjustment of the pH and finally preparation of the hydrogel.

### Overview of the Invention

The invention provides for a method for decellularizing corneal extracellular matrix by treating corneal extracellular matrix with one or more decellularizing agent selected from a group comprising sodium dodecyl sulfate, Triton X-100, sodium chloride, sodium deoxycholate, 3-[(3-Cholamidopropyl) dimethylammonio]-1-propanesulfonate, trypsin, EDTA, sulfobetaines-10, sulfobetaines-16, nuclease, protease, collagenase, lipase, thermolysin, α-galactosidase, Tri(n-butyl) phosphate, glycerol, isopropanol, ethanol and methanol; and further, treating the corneal extracellular matrix with RNase and DNAase.

In one embodiment, the concentration of sodium dodecyl sulfate or Triton X-100 is in a range from 0.1% to 3%. Sodium chloride is present at a concentration in a range from 0.1% to 3%.

In another embodiment, the concentration of RNase used in the method is at a range from 0.5 U to 50 U/mL and the concentration of DNase is at a range from 1 U/mL to 200 U/mL.

In yet another embodiment, the source of the corneal extracellular matrix is selected from a group comprising human source, caprine source, porcine source or bovine source.

The invention also provides for a method for preparation of hydrogel based on corneal extracellular matrix by decellularizing corneal extracellular matrix, digesting the decellularized corneal extracellular matrix, adjusting the pH of the digested corneal extracellular matrix to obtain pre-gel, and mixing one or more cell culture and suitable culture media with the pre-gel to obtain hydrogel.

In one embodiment, the decellularized corneal extracellular matrix is digested using acetic acid and pepsin. In another embodiment, the concentration of acetic acid is in the range from 0.1 M to 2 M. In yet another embodiment, the weight of pepsin is in the range from 10% to 15% of the total weight of the decellularizing corneal extracellular matrix.

In a further embodiment, the pH of the digested corneal extracellular matrix is adjusted at a temperature below 4° C.

In another embodiment, the culture media is selected from a group comprising α-MEM, DMEM, RPMI-1640, Media-199 and Ham's F-12. In another embodiment, the cell culture is selected from a group comprising keratocytes, epithelial cells, limbal stem cells, stromal stem cells, endothelial cells and retinal pigment epithelial cells.

The invention also provides a method of treating corneal diseases or disorders by administration of hydrogel obtained in accordance with one embodiment of the invention.

### BRIEF DESCRIPTION OF DRAWINGS

FIG. 1 depicts the caprine cornea used for further processing and preparation of hydrogel.

FIG. 2 depicts minced stromal layer of the caprine cornea. Minced pieces of the stromal layer are about 2-3 mm in size.

FIG. 3 depicts decellularized minced stromal layer of caprine cornea, obtained after subjecting the minced pieces to the process of decellularization.

FIG. 4 depicts lyophilized corneal matrix which can be stored for further usage.

FIG. 5 depicts the results of the picogreen assay.

FIG. 6 depicts histological analysis of (A) Native cornea. (B) Decellularized cornea, stained with haematoxylin and eosin.

FIG. 7 depicts the process for preparing the hydrogel.

FIG. 8 depicts the final corneal hydrogel for application.

FIG. 9 depicts native corneal sample after staining with haemotoxylin and eosin (H&E).

FIG. 10 depicts decellularized corneal sample after staining with haemotoxylin and eosin (H&E).

FIG. 11 depicts the final injectable hydrogel after adjusting the pH and addition of PBS.

FIG. 12 depicts the final injectable hydrogel at varied time intervals.

FIG. 13 and FIG. 14 depicts intra-lamellar pockets created using the crescent blade.

FIG. 15 shows injection of 100 μl of corneal hydrogel into intra-lamellar pocket of the cadaveric corneal samples.

FIG. 16 depicts injected hydrogel in lamellar pockets.

FIG. 17 depicts the corneal sample after successful injection.

FIG. 18 shows the incubation of the cadaveric human eyes in DMEM media.

FIG. 19, FIG. 20, FIG. 21, FIG. 22 and FIG. 23 depicts stages of dissection of corneal sample for harvesting corneal hydrogel.

FIG. 24 depicts staining of dissected corneal sample with Hoechst® and rhodamine phalloidin.

FIG. 25 and FIG. 27 depicts transverse section of cornea without hydrogel.

FIG. 26 and FIG. 28 depicts transverse section of cornea with hydrogel.

FIG. 29 depicts an enlarged view of a part of the morphology of the transverse section of cornea with hydrogel.

FIG. 30 depicts cornea with hydrogel where migrated cells are visible with different morphology.

FIG. 31 depicts the results of cell viability assay.

FIG. 32 depicts the results of the scratch assay.

### DEFINITIONS

Unless defined otherwise, all technical and scientific terms used herein have the same meaning as commonly understood by one of ordinary skill in the art to which the methods belong. Although any device, kit, method and composition similar or equivalent to those described herein can also be used in the practice or testing of the methods and compositions, representative illustrative methods and compositions are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

As used herein, the term “hydrogel” or “bioink” refers to a material that behaves much like a semi solid, with pH adjusted corneal decellularized and digested extracellular matrix as major composition allowing for creation of a desired shape. Hydrogel or bioink is typically a liquid composition, which may contain living cells. Bioink may also be a liquid suspension of the cells or growth factors. The cells may for instance be suspended in cell culture medium, or buffered solution. The hydrogel or bioink may comprise one or more reagent along with the cell culture medium. The bioink may further comprise a carrier suitable for cells or a solvent. The solvent typically comprises water. The carrier may be cell culture medium or buffered solution. Bioink or hydrogels are prepared by methods described herein.

As used herein, the term “neutral buffered solution” refers to refers to a compound, usually salts, which, when dissolved in an aqueous medium, serves to maintain the free hydrogen ion concentration of the solution within a certain pH range, usually between 5-9 when hydrogen ions are added or removed from the solution.

As used herein, the term “minced” or “mincing” refers to the process in which a sample of biological tissue that has been chopped, ground, sliced, cut, worked into a paste or otherwise reduced in minimum particle size from the native tissue state to having particles no larger than about 2-3 mm in size. The minced tissue contains tissue fragments, clumps or clusters of cells, individual whole cells, and may also contain a portion of ruptured cells.

As used herein, the term “decellularization” refers to the removal of cells from tissue or organs. Preferably, decellularization may be carried out with minimum possible damage to the structure and function of the original tissue or organs.

As used herein, the term “corneal extracellular matrix” refers to whole or part of cornea which can be used for preparation of hydrogel. The components of corneal extracellular matrix include, but are not limited to, collagens and proteoglycans. Sources of corneal extracellular matrix can be both human and animal sources such as, but not limited to, domestic large and small animals such as dogs, cats, rabbits, horses, cows, pigs, and the like.

As used herein, the term “digesting” refers to breaking down or degradation of proteins into smaller peptides.

As used herein, the term “pre-gel” refers to decellularized, digested and pH-adjusted corneal extracellular matrix as described herein.

### DETAILED DESCRIPTION OF THE INVENTION

The present invention discloses methods for preparation of hydrogels based on decellularized corneal matrix and hydrogels for treatment of corneal and defects and diseases.

The invention contemplates a multidimensional approach in development of highly efficacious hydrogels by adopting a modified process for decellularization of corneal matrix and further conversion of the decellularized matrix into hydrogel. The hydrogels produced by the method are extremely useful as injectable gels for treatment of various pathological conditions pertaining to cornea.

The hydrogel formulated in the present invention can be injected into the corneal stromal region as treatment strategy to deal with keratoconus. This can be achieved by thickening the stromal area by using a novel hydrogel which integrates with the existing thin stroma and upon longer time period the filled area become transparent with the cues from microenvironment.

Further, the invention also relates to method of treating corneal defects using hydrogels developed in this invention.

The highly efficacious nature of the decellularized corneal extracellular matrix and hydrogels are exhibited by the following:
Low left-over DNA content in decellularized matrix exhibited by Picogreen assay (Example 3)
Absence of cellular debris or nuclei in decellularized matrix (Example 4)
High retention of collagen and glycosaminoglycans (Example 5)
High integrity and transparency of the hydrogel (Example 10)
Increase in corneal thickness (Example 11)
Cell viability is high in the hydrogel (Example 12)
High cell migration as exhibited by scratch assay (Example 13)

Before the methods and hydrogels of the present disclosure are described in greater detail, it is to be understood that the invention is not limited to particular embodiments and may vary. It is also to be understood that the terminology used herein is for the purpose of describing particular embodiments only, and is not intended to be limiting, since the scope of the methods and compositions will be limited only by the appended claims.

The present invention discloses methods for preparing decellularized corneal matrix-based hydrogel and bioink compositions.

For the first time, the inventors have used decellularized corneal samples for preparation of a novel hydrogel. The inventors have devised a novel method for processing the decellularized corneal samples to obtain a novel hydrogel which can be used in a wide range of medical conditions pertaining to cornea.

The process for preparation of the hydrogel involves four major steps:

i. Decellularization of the corneal samples

ii. Digestion of the decellularized corneal sample

iii. Adjustment of pH for preparation to obtain final pre-gel

iv. Preparation of hydrogel (bioink)

The inventors have also devised a method for preparation of bioink using the hydrogel of the present invention.

The inventors have identified the critical process parameters which is required for the preparation of the hydrogel. The hydrogel prepared is highly efficacious and exhibits advantageous properties as against hydrogels known in the art. Further, the hydrogel is highly inexpensive and can be afforded by people in developing and less developed countries.

The present invention represents an advancement over the existing art for preparation of hydrogel for use in corneal diseases.

In one embodiment of the invention, corneal samples are collected from slaughter houses or tissue banks. The corneal samples collected can be from different animals including, but not limited to, caprine, bovine, porcine or human corneal samples.

In another embodiment, the corneal samples are cadaveric corneal samples.

In another embodiment, the stromal layers of the corneal samples are isolated from the whole caprine cornea. The isolated stromal layer was processed by mincing into small pieces.

In another embodiment, stromal layers are of the size of about 2-3 mm.

Any mincing or cutting or shredding method known to person having skill in the art can be employed for the purpose of reducing the size of the stromal layer to the desired size.

In one embodiment of the invention, methods for decellularization of corneal extracellular matrix is provided.

In another embodiment of the invention, decellularization of corneal extracellular matrix is done using one or more decellularizing agent selected from a group comprising sodium dodecyl sulfate, Triton X-100, sodium chloride, sodium deoxycholate, 3-[(3-Cholamidopropyl) dimethylammonio]-1-propanesulfonate, trypsin, EDTA, sulfobetaines-10, sulfobetaines-16, nuclease, protease, collagenase, lipase, thermolysin, α-galactosidase, Tri(n-butyl) phosphate, glycerol, isopropanol, ethanol and methanol.

In another embodiment, sodium dodecyl sulfate is used for decellularization process.

In another embodiment, the concentration of sodium dodecyl sulfate is in a range from 0.1% to 3%.

In another embodiment, Triton X-100 is used for decellularization process.

In another embodiment, the concentration of Triton X-100 is in a range from 0.1% to 3%.

In another embodiment, sodium chloride is used for decellularization process.

In another embodiment, the concentration of sodium chloride is in a range from 0.5 M to 5 M.

In another embodiment distilled water or phosphate-buffered saline (pBS) is used for decellularization.

In a further embodiment, the corneal matrix with decellularizing agents for a period in the range of 12-72 hrs for decellularization.

In another embodiment of the invention, the corneal extracellular matrix is treated with DNase or RNase for removal of DNA or RNA content.

In one embodiment the concentration of RNase is in the range from 0.5 U/mL to 50 U/mL.

In another embodiment the concentration of DNase is in the range from 1 U/mL to 200 U/mL.

The treatment with DNase or RNase is done for a period in the range of 4 hrs to 8 hrs at a temperature in the range of 10-40° C.

In another embodiment, the decellularized corneal tissue were then washed thrice in distilled water or phosphate-buffered saline (pBS) for 1 to 3 days for removal of detergent.

In a further embodiment, the decellularized tissues were treated with 0.01%-1% peracetic acid in 1% to 10% ethanol for 1 to 10 hours followed by washing several times with PBS solution.

In a further embodiment, the decellularized corneal tissue is lyophilized at temperature range of −10° C. to −90° C. using standard techniques and stored at a temperature in the range of −4° C. to −90° C. for further use.

In a further embodiment, the decellularized corneal matrix is crushed into powder using a mortar and pestle with the help of liquid nitrogen for digestion. Alternatively, the decellularized corneal matrix is pulverized into powder by using a mill.

In another embodiment, the required amount of decellularized corneal matrix is taken and digested with digestion solution comprising acetic acid and pepsin.

In one embodiment, the concentration of acetic acid is in the range of 0.1 M to 2 M.

In a further embodiment, the weight of pepsin is in the range of 5%-15% of the weight of the total weight of the tissue.

In another embodiment, a digestion solution comprising 0.01-0.5 N HCl and 5-15% by weight pepsin is used for the digestion process.

In further embodiments, any suitable digestion enzyme can be employed for the purpose of digesting the decellularized corneal matrix.

In another embodiment, the pH of the hydrogel is adjusted by dropwise addition of any basis solution to bring the pH of the solution in a range from 7-8.

In another embodiment, the temperature was maintained below 4° C. to avoid gelation of the decellularized corneal matrix.

In another embodiment, a hydrogel is prepared by allowing the pre-gel to form a gel at a temperature in the range of 34-38° C. for about 30-45 minutes.

In further embodiments, antibiotics, stabilizers, additives and preservatives can be added to the prepared hydrogel for final application.

In another embodiment, 0.5-5% pH-adjusted decellularized corneal matrix pre-gel is taken and 5-15% of 10× suitable culture media is added.

In further embodiments, required growth factors and/or enzymes are also added.

In further embodiments, suitable culture medium is selected from a group comprising, but not limited to, α-MEM, DMEM, RPMI-1640, Media-199 and Ham's F-12 media. In essence, any cell culture media which can provide nutrients for the requirement of cells is used for preparation of the hydrogel (bioink).

In further embodiments, required growth factors/enzymes are selected from a group comprising, but not limited to, bFGF.VEGF, EGF, TIMPs, MMPs. In essence, any low molecular weight proteins which can act in vitro by mimicking the invivo conditions to provide requirement of cells is used for preparation of the bioink.

In further embodiments, cell cultures are mixed with pH adjusted pre-gel and subjected to gelation at a temperature in the range of 34° C.-38° C. for about 30-45 minutes to obtain the final hydrogel (bioink).

Suitable cells for mixing with the pH adjusted pre-gel is selected from a group comprising keratocytes, epithelial cells, limbal stem cells, endothelial cells and retinal pigment epithelial cells.

In another embodiment, hydrogel (bioink) prepared in the invention optionally comprises one or more preservatives.

In another embodiment, hydrogel (bioink) prepared in the invention optionally comprises one or more carriers.

In another embodiment, hydrogel (bioink) prepared in the invention optionally comprises one or more excipients.

In one embodiment, the hydrogel (bioink) can be suitably used for 3-D bioprinting of implants and in vitro healthy as well diseased models.

In other embodiments, the hydrogel (bioink) can also be used for treatment of corneal diseases or disorders.

In further embodiments, the hydrogel (bioink) can be used for treatment of keratoconus.

In other embodiments, the hydrogel can be used for treatment corneal ulceration which leads to descemetocele formation.

In other embodiments, the hydrogel can be uses as filler for corneal perforations.

In further embodiments, the hydrogel can be used for corneal perforations resulting from penetrating, chemical or surgical trauma. In another embodiment, the hydrogel can be used for treatment of corneal scar resulting from abrasion, laceration, burns or diseases.

### EXAMPLES

Before the compositions and methods of the present disclosure are described in greater detail, it is to be understood that the invention is not limited to particular embodiments and may vary. It is also to be understood that the terminology used herein is for the purpose of describing particular embodiments only and is not intended to be limiting.

### Example 1: Collection and Processing of Corneal Samples

Cadaveric caprine corneal samples were collected from slaughter houses (Hyderabad, India) with appropriate approvals. The corneal samples collected can be from different animals including, but not limited to, caprine, bovine, porcine or human corneal samples.

FIG. 1 depicts cadaveric caprine corneal sample used for preparation of hydrogel.

The stromal layers of the corneal samples were isolated from the whole caprine cornea. The isolated stromal layer was processed by mincing into small pieces of about 2-3 mm.

Any mincing or cutting or shredding method known to person having skill in the art can be employed for the purpose of reducing the size of the stromal layer to the desired size.

FIG. 2 depicts minced stromal layer of the caprine cornea. Minced pieces of the stromal layer are about 2-3 mm in size.

### Example 2: Decellularization of Cadaveric Cornea

The minced stromal layer was then subjected to the process of decellularization by stirring in 1% sodium dodecyl sulfate in a neutral buffered solution. 0.1-3% sodium dodecyl sulfate can be used for the decellularization process. Alternatively, distilled water or phosphate-buffered saline (pBS) can be used for decellularization. The minced stromal layers were stirred for a period of 48 hours. The stirring can be done between 12-72 hrs.

It was further followed by treatment with 1% Triton-X-100 solution for 24 hours. The concentration of Triton-X solution can be in the range of 0.1%-3% and treatment can be done for a period in the range of 12-72 hrs.

Alternatively, the inventors have modified the decellularization steps by treating the minced stromal layer with 1.5 M sodium chloride solution in distilled water or phosphate-buffered saline (pBS) for 48 hours. The concentration of sodium chloride solution can be in the range of 0.5 M to 5 M and treatment can be done for a period in the range of 12-72 hrs.

Further, steps for decellularization involves treatment with RNase having concentration of 1 U/mL and DNase having concentration of 50 U/mL for a period of 4 hrs. 4-8 hrs. RNase having concentration in the range of 0.5-50 U/mL and DNase having concentration in the range of 1-200 U/mL can be used. Further, the stromal layer can be treated for a period of 4-8 hrs. The decellularization process in carried out at room temperature. The temperature can be varied in the range of 10-40° C.

The decellularized corneal tissue were then washed thrice in distilled water or phosphate-buffered saline (pBS) for 1 to 3 days for removal of detergent.

The decellularized tissues were treated with 0.1% peracetic acid in 4% ethanol for 4 hours followed by washing several times with PBS solution. 0.01% to 1% peracetic acid can be used which can be diluted in 1% to 10% ethanol and the treatment can be done for 1 to 10 hours.

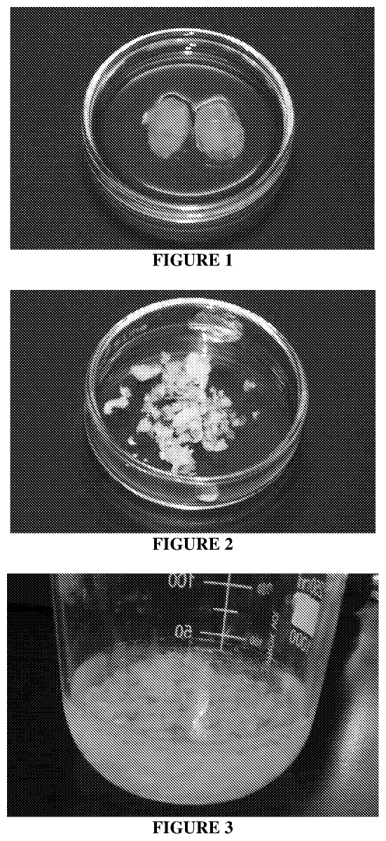

FIG. 3 depicts decellularized minced stromal layer of caprine cornea, obtained after subjecting the minced pieces to the process of decellularization.

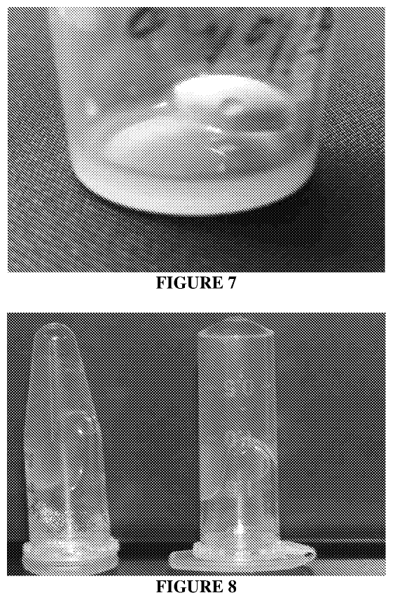

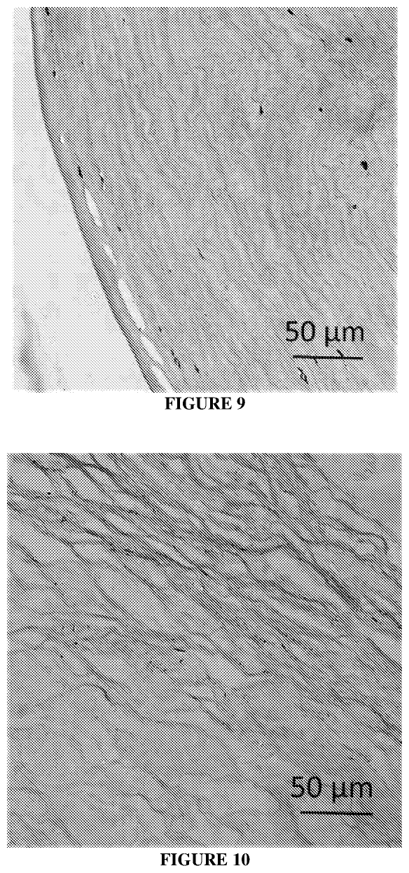

FIG. 7 depicts native corneal sample and FIG. 8 depicts decellularized corneal sample after staining with haemotoxylin and eosin (H&E).

The decellularized corneal tissue was further lyophilized at −80° C. using standard techniques and stored at −20° C. for further use. The decellularized matrix can be lyophilized at a temperature range of −10° C. to −90° C. and it can be stored at a temperature in the range of −4° C. to −90° C.

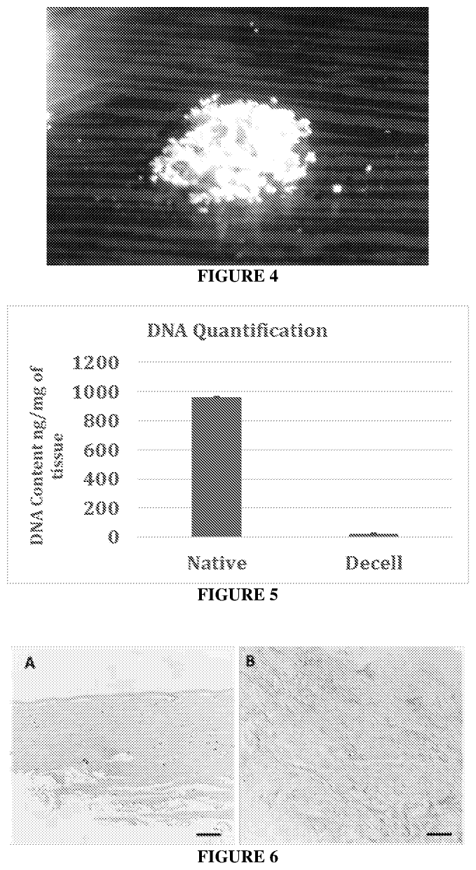

FIG. 4 depicts lyophilized corneal matrix which can be stored for further usage.

### Example 3: Quantification of DNA Content in Decellularized Corneal Matrix Using Picogreen Assay

The DNA content in hydrogel was detected and quantified using standard Picogreen assay for quantifying the leftover DNA content. It was observed that left over DNA content in decellularized corneal matrix was 22 ng/mg of tissue and that of native was 962 ng/mg of tissue. The results are depicted in FIG. 5.

TABLE 1
Leftover DNA content

| Sample | Leftover DNA content |
|---|---|
| Native tissue | 962 ng/mg |
| Decellularized corneal matrix | 22 ng/mg |

### Example 4: Histology of Native and Decellularized Corneal Matrix

The histology of the native and decellularized corneal matrix were checked to assess the extent of decellularization. The samples were fixed in 4% formaldehyde for 4 hrs, washed with PBS, dehydrated in graded alcohol series and subsequently embedded in paraffin.

For histological staining, 5 mm thick sections were stained with hematoxylin and eosin (H&E) and examined under a light microscope. It was observed that the corneas have been decellularized successfully as evidenced by the absence of cellular debris or nuclei in the dECM histology images in FIG. 6.

### Example 5: Collagen and Glycosaminoglycans (GAGs) Assay

The collagen and glycosaminoglycan content of the decellularized corneas were checked by performing collagen and glycosaminoglycan assay. It was observed that around 140% of GAGs and 78% total collagen were retained in the decellularized tissue compared to native corneal tissue, which revealed that high quantity of decellularized extracellular matrix components are being preserved after decellularization.

### Example 6: Digestion of Corneal Samples

For digestion of the decellularized corneal tissue, the decellularized matrix was crushed into powder using a mortar and pestle with the help of liquid nitrogen.

The required amount of decellularized corneal matrix was taken and digested with digestion solution comprising acetic acid and pepsin. The concentration of acetic acid is 0.5 M, which can be in the range of 0.1 M to 2 M. The weight of pepsin is 10% of the weight of the total weight of the tissue. The weight of pepsin can be in the range of 5%-15% of the total weight of the tissue. Accordingly, 10 mg of pepsin was required for digesting 100 mg of decellularized corneal matrix.

Alternatively, a digestion solution comprising 0.01 N HCl and 5-15% by weight pepsin was used for the digestion process. Any suitable digestion enzyme can be employed for the purpose of digesting the decellularized corneal matrix.

### Example 7: Adjustment of pH

The pH of the hydrogel was checked and adjusted by dropwise addition of cold 10M NaOH solution to 7.4. Alternatively, any basic solution can be used for adjusting the pH of the solution to a range of 7-8.

The temperature was maintained below 4° C. to avoid gelation of the decellularized corneal matrix. The pH adjusted decellularized corneal matrix pre-gel is stored at 4° C. for further use.

The pre-gel is further optimized at a temperature in the range of 34-38° C. for about 30-45 minutes for obtaining the final hydrogel.

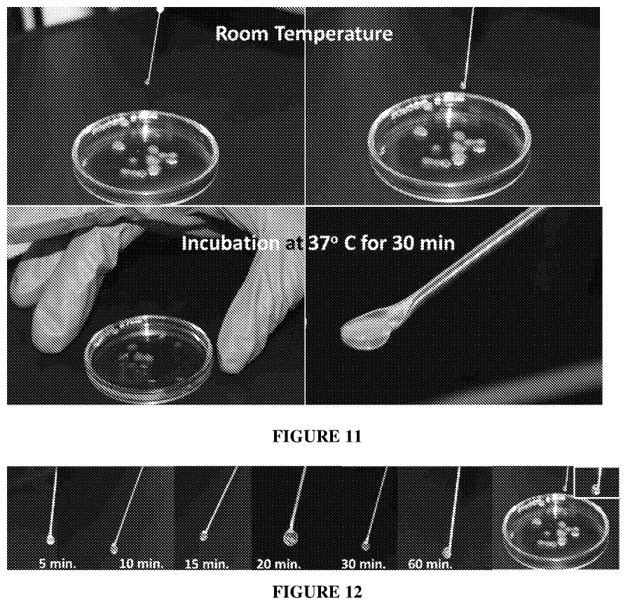

FIG. 7 depicts the process for preparing the hydrogel. FIG. 8 depicts the final corneal hydrogel for application. FIG. 11 depicts the final injectable hydrogel after adjusting the pH and addition of PBS. FIG. 12 depicts the final injectable hydrogel at varied time intervals.

Further, antibiotics, stabilizers, additives and preservatives can be added to the prepared hydrogel for final application.

### Example 8: Preparation of Hydrogel (Bioink)

3% of pH-adjusted decellularized corneal matrix pre-gel was then taken and 10% of 10× of suitable culture media. Optionally, required growth factors and/or enzymes are also added. As per requirements, 0.5-5% pH-adjusted decellularized corneal matrix pre-gel is taken and 5-15% of 10× suitable culture media is added.

Suitable culture medium is selected from a group comprising, but not limited to, α-MEM, DMEM, RPMI-1640, Media-199 and Ham's F-12 media. In essence, any cell culture media which can provide nutrients for the requirement of cells is used for preparation of the bioink.

Required growth factors/enzymes are selected from a group comprising, but not limited to, bFGF.VEGF, EGF, TIMPs, MMPs. In essence, any low molecular weight proteins which can act in vitro by mimicking the invivo conditions to provide requirement of cells is used for preparation of the bioink.

Subsequently, cells are mixed with this pH adjusted pre-gel and the formed bioink is subjected to gelation at a temperature in the range of 34° C.-38° C. for about 30-45 minutes to obtain the final bioink.

Suitable cells for mixing with the pH adjusted pre-gel is selected from a group comprising keratocytes, epithelial cells, limbal stem cells, endothelial cells and retinal pigment epithelial cells.

The bioink resulting from hydrogels can be suitably used for 3-D bioprinting of implants and in vitro healthy as well diseased models.

### Example 9: Intra-Lamellar Injection of Corneal Hydrogel into Cadaveric Human Cornea

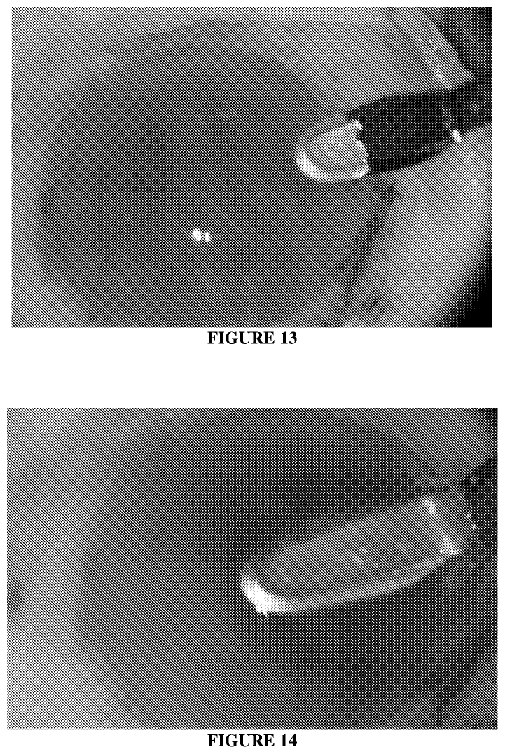

Human cadaveric eyes (collected from a 95-year old female body at LV Prasad Eye Institute, Hyderabad) was used in the process. Intra-lamellar pockets were created in the corneas of the cadaveric eyes by a crescent blade using standard procedure. FIG. 13 and FIG. 14 depicts intra-lamellar pockets created using the crescent blade.

Subsequently, the hydrogel obtained was injected to the intra-lamellar pockets through standard intra-lamellar injection procedure.

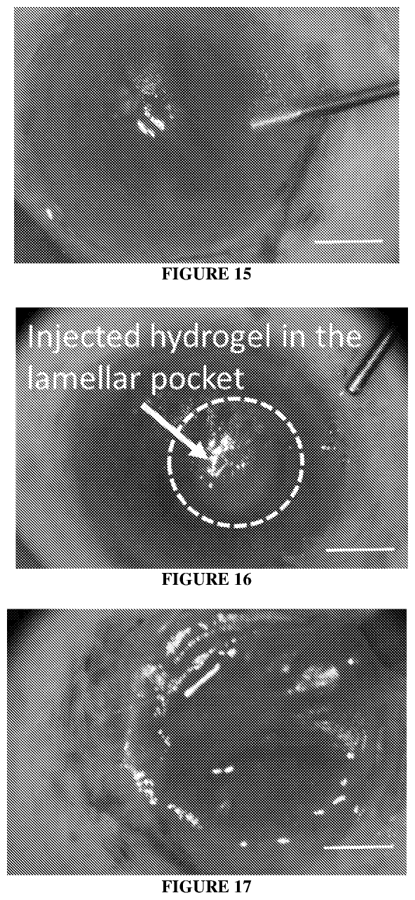

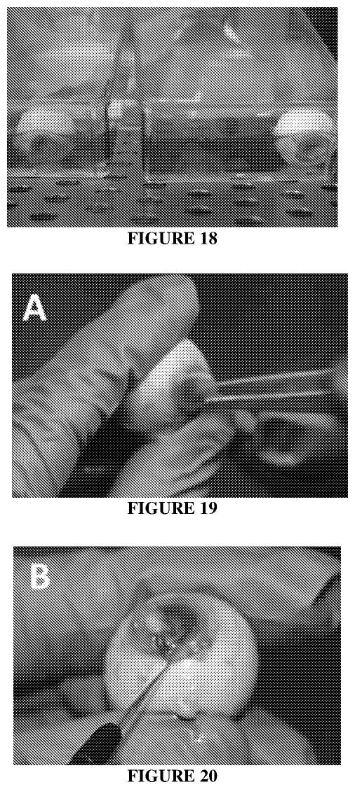

FIG. 15 shows injection of 100 μl of corneal hydrogel into intra-lamellar pocket of the cadaveric corneal samples. FIG. 16 depicts injected hydrogel in lamellar pockets and FIG. 17 depicts the corneal sample after successful injection.

### Example 10: Evaluation of Cell Migration from the Corneal Stroma to the Injected Hydrogel

Thereafter, an assessment was performed for studying cellular migration to the injected hydrogel. The cadaveric human eyes were incubated in Dulbecco's Modified Eagle's Medium (DMEM) at 37° C. and 5% CO₂ for 14 days.

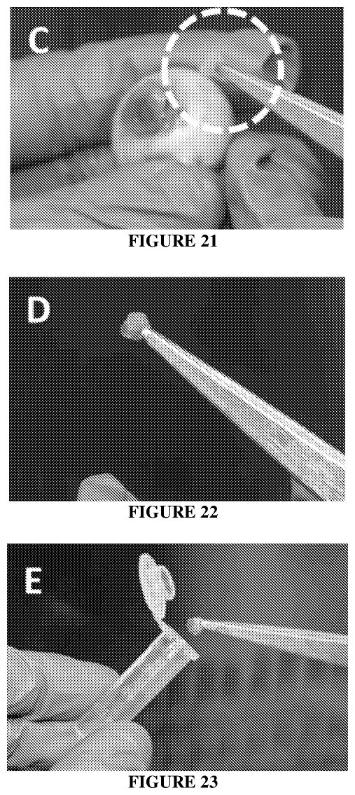

FIG. 18 shows the incubation of the cadaveric human eyes in DMEM media.

After 14 days, the injected corneal hydrogel was harvested by dissection. The stages of dissection are depicted in FIG. 19, FIG. 20, FIG. 21, FIG. 22 and FIG. 23.

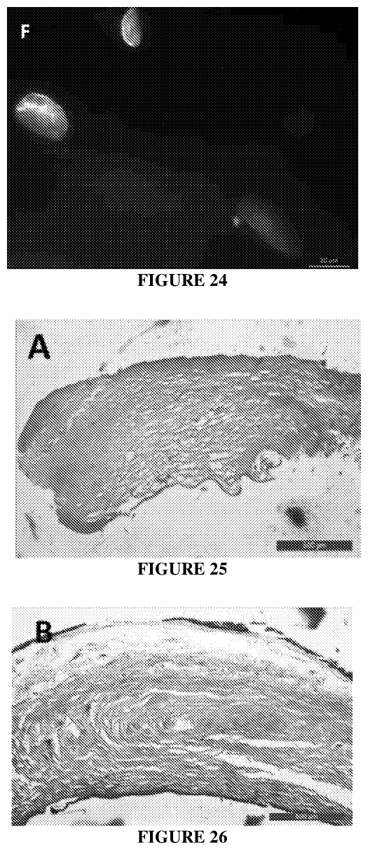

The dissected corneal sample was stained with Hoechst®, a blue fluorescent stain specific for nucleic DNA and rhodamine phalloidin, a high-affinity F-actin probe conjugated to the red-orange fluorescent dye, tetramethylrhodamine (TRITC). The results are depicted in FIG. 24.

The confocal study with fluorescent staining revealed that the cells from the stroma of cadaveric human cornea migrated to the injected corneal hydrogel. The migrating cells to the hydrogel results in integration and transparency of the injected hydrogel under in vivo condition.

### Example 11: Evaluation of Corneal Thickness after Injection of Hydrogel

Corneal thickness was measured after 14 days of injecting the hydrogel. All the measurements were taken from epithelial layer to endothelial layer. Both the corneas used were normal cornea not exhibiting any pathological conditions such as keratinous cornea.

TABLE 2
Evaluation of corneal thickness after injection of hydrogel

| Corneal Sample | Thickness |
|---|---|
| Cornea without hydrogel | 767 ± 71 μm |
| Cornea with hydrogel | 1271 ± 57 μm |

It was observed that there is an increase of about 504 μm in the thickness of the cornea.

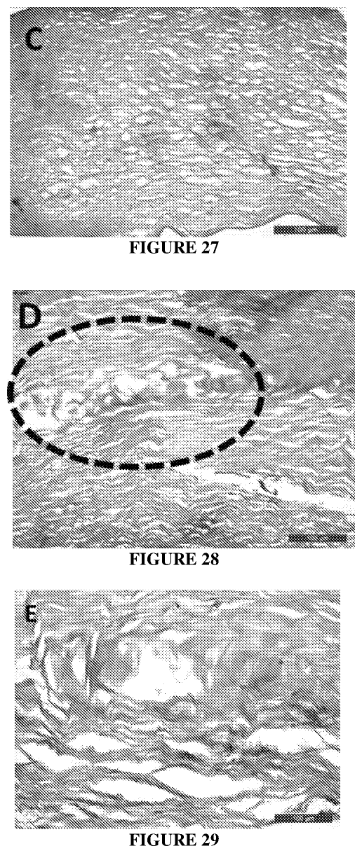

The corneal samples with injected hydrogel was fixed and histology was performed on the same by staining with haemotoxylin and eosin (H&E). FIG. 25 and FIG. 27 depicts transverse section of cornea without hydrogel.

FIG. 26 and FIG. 28 depicts transverse section of cornea with hydrogel. FIG. 29 depicts an enlarged view of a part of the morphology of the transverse section of cornea with hydrogel.

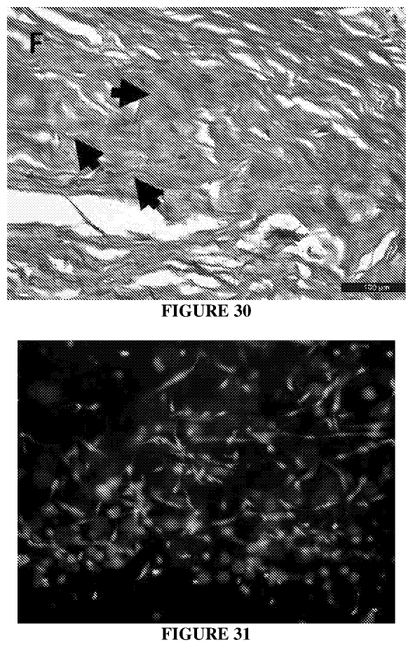

FIG. 30 depicts cornea with hydrogel where migrated cells are visible with different morphology.

Therefore, the injected hydrogel can contribute to the thickness of cornea and also results in integration and transparency of the injected hydrogel under in vivo condition.

### Example 12: Cell Viability Assay

The cell viability assay was done to determine whether the hydrogel contains any compounds or molecules, which have adverse effects on cell proliferation or causes cell death.

The live-dead assay format was chosen to check cell viability, which is a two-color assay to determine the viability based on the plasma membrane integrity.

Hydrogels containing stromal cells were checked after 10 days for cell viability. It was observed that majority of the stromal cells in hydrogel is viable and attaining typical spindle shape morphology.

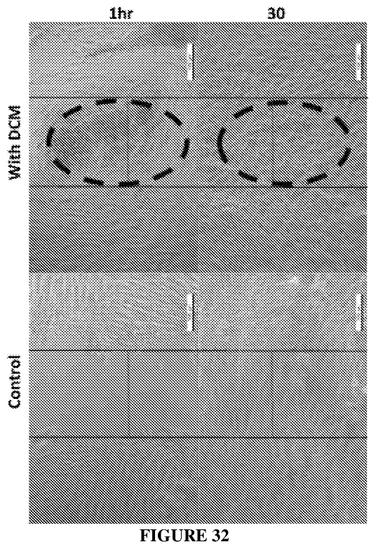

The results are depicted are in FIG. 31.

### Example 13: Scratch Assay

An in vitro scratch assay was performed to find out the cell migration in presence of decellularized corneal matrix. The scratch assay method is based on the observation that, upon creation of a new artificial gap (scratch) on a confluent cell monolayer, the cells on the edge of the newly created gap will move toward the opening to close the scratch until new cell-cell contacts are established again.

The assay was performed by creating a gap on a confluent layer of human corneal stromal cells (human keratocyte) manually using p 200 pipette tip. The gap was filled with a thin layer of decellularized corneal matrix pre-gel, which formed hydrogel after some time. A control was also set up which did not contain the decellularized corneal matrix pre-gel.

Images were captured until cell-cell contact was re-established. It was observed that cells were migrating towards the gap in both control and test samples. Cells could bridge the gap in both the conditions within 30 h of incubation. In addition, there was a swarming growth of cells towards the gel from the surrounding area towards the gel, which was absent in the control sample.

The results are depicted in FIG. 32.

1. A method for decellularizing corneal extracellular matrix, comprising the steps of:
a. treating corneal extracellular matrix with one or more agents selected from a group comprising sodium dodecyl sulfate, Triton X-100, sodium chloride, sodium deoxycholate, 3-[(3-Cholamidopropyl) dimethylammonio]-1-propanesulfonate, trypsin, EDTA, sulfobetaines-10, sulfobetaines-16, nuclease, protease, collagenase, lipase, thermolysin, α-galactosidase, Tri(n-butyl) phosphate, glycerol, isopropanol, ethanol and methanol; and
b. treating the corneal extracellular matrix obtained in step (b) with RNase and DNase to obtain decellularized corneal extracellular matrix.

2. The method as claimed in claim 1, wherein sodium dodecyl sulfate or Triton X-100 is present at a concentration in a range from 0.1% to 3%.

3. The method as claimed in claim 1, wherein sodium chloride is present at a concentration in a range from 0.1% to 3%.

4. The method as claimed in claim 1, wherein the concentration of RNase is in a range from 0.5 U/mL to 50 U/mL.

5. The method as claimed in claim 1, wherein the concentration of DNase is in a range from 1 U/mL to 200 U/mL.

6. The method as claimed in claim 1, wherein source of the corneal extracellular matrix is selected from a group comprising human source, caprine source, porcine source or bovine source.

7. Decellularized corneal extracellular matrix obtained by a method as claimed in claim 1.

8. A method for preparation of hydrogel based on corneal extracellular matrix, comprising the steps of:
a. decellularizing corneal extracellular matrix by a method as claimed in claim 1;
b. digesting the decellularized corneal extracellular matrix using acetic acid and pepsin;
c. adjusting the pH of the digested corneal extracellular matrix in a range from 7 to 8 at a temperature below 4° C. to obtain pre-gel; and
d. mixing one or more cell culture and suitable culture media with the pre-gel obtained in step (c) to obtain hydrogel.

9. The method as claimed in claim 8, wherein the concentration of acetic acid is in a range from 0.1 M to 2 M and weight of pepsin is in a range from 5% to 15% of the total weight of the decellularizing corneal extracellular matrix.

10. The method as claimed in claim 8, wherein the culture media is selected from a group comprising α-MEM, DMEM, RPMI-1640, Media-199 and Ham's F-12.

11. The method as claimed in claim 8, wherein the cell culture is selected from a group comprising keratocytes, epithelial cells, limbal stem cells, stromal stem cells, endothelial cells and retinal pigment epithelial cells.

12. Hydrogel obtained by a method as claimed in claim 8.

13. Hydrogel as claimed in claim 12, optionally comprising one or more preservatives, carriers or excipients.

14. Agents selected from a group comprising sodium dodecyl sulfate, Triton X-100 and sodium chloride for use in decellularization of corneal extracellular matrix.

15. Hydrogel as claimed in claim 12 for use in treatment of corneal diseases or disorders.

16. A method of treating corneal diseases or disorders, comprising administration of hydrogel as claimed in claim 12.
